(19) United States
(12) Patent Application Publication
(10) Pub. No.: US 2024/0358527 A1
(43) Pub. Date: Oct. 31, 2024

(54) DEVICES FOR BIOMECHANICAL ASSISTANCE INCLUDING AN EXOSKELETON AND A ROBOTIC GLOVE

(71) Applicant: UNIVERSITY OF NORTH TEXAS, Dallas, TX (US)

(72) Inventors: Emilly HAYS, Dallas, TX (US); Emily CARNEY, Dallas, TX (US); Kobe CRUZ, Dallas, TX (US); Jack SLAYTON, Dallas, TX (US); Gary TEJEDA-GODINEZ, Dallas, TX (US); Trevor W. EXLEY, Dallas, TX (US); Amir JAFARI, Dallas, TX (US)

(21) Appl. No.: 18/649,708

(22) Filed: Apr. 29, 2024

Related U.S. Application Data
(60) Provisional application No. 63/498,739, filed on Apr. 27, 2023.

(51) Int. Cl. A61F 2/54

(52) U.S. Cl. CPC A61F 2/54; A61F 2220/0075

(57) ABSTRACT

A device can include at least one of an exoskeleton comprising a preloaded elastic spring, a plurality of drums coupled to the preloaded elastic spring, and a cable coupled to a user, the plurality of drums and the preloaded elastic spring; and a robotic hand. The robotic hand includes a glove comprising digits, a tubing system in fluid communication with a fluid supply, a plurality of sensors coupled to the digits of the glove, and a plurality of soft actuators coupled to the tubing system and the plurality of sensors. The plurality of soft actuators comprises a thickened base, a fluid cavity above the thickened base defined by a tube, and a retaining device wrapped around the tube.

### CROSS-REFERENCE TO RELATED APPLICATIONS

This application claims the benefit of U.S. Provisional Application No. 63/498,739, filed on Apr. 27, 2023, and entitled “HYDRAULIC-ACTUATED SOFT ROBOTIC GLOVE”, which is incorporated herein by reference in its entirety for all purposes.

### STATEMENT REGARDING FEDERALLY SPONSORED RESEARCH OR DEVELOPMENT

This invention was made with government support under contract no. 2213263 awarded by The National Science Foundation. The government has certain rights in the invention.

### BACKGROUND

In the USA alone, more than twenty million Americans have an impairment that affects at least one of their limbs. Many people experience various limb impairments including paralysis, cerebral palsy, stroke, or multiple sclerosis. Thus, there is a need for devices that improve their biomechanical capabilities.

### SUMMARY

In some embodiments, a device can include an exoskeleton including a preloaded elastic spring; a plurality of drums coupled to the preloaded elastic spring; and a cable coupled to a user, the plurality of drums and the preloaded elastic spring; and a robotic hand.

In some embodiments, an exoskeleton can include a preloaded elastic spring; a plurality of drums coupled to the preloaded elastic spring; and a cable coupled to a user, the plurality of drums and the preloaded elastic spring.

In some embodiments, a robotic hand can include a glove comprising digits; a vinyl tubing system in fluid communication with a fluid supply; a plurality of sensors coupled to the digits of the glove; and a plurality of soft actuators coupled to the plurality of sensors. The plurality of soft actuators can include a thickened base, a fluid cavity above the thickened base defined by a tube, and a retaining device wrapped around the tube.

In some embodiments, a device can increase a user's grip strength and range of motion. Desirably, the device is relatively lightweight.

In some embodiments, a method of movement comprises receiving a force input on a sensor of a plurality of sensors on a glove, actuating a pump in response to the force input, wherein the pump is in fluid communication with the fluid supply, transferring fluid between the fluid supply and at least one soft actuator of the plurality of soft actuators, and applying a force to at least one digit of the plurality of digits in response to the transfer of the fluid. The glove comprises a plurality of digits, a tubing system in fluid communication with a fluid supply, the plurality of sensors coupled to the digits of the glove, and a plurality of soft actuators coupled to the tubing system and the plurality of sensors.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 is an elevational, schematic view of an embodiment of a device including an exoskeleton.

FIG. 2 is a schematic view of an embodiment of a plurality of drums coupled to a preloaded elastic spring.

FIG. 3 is a block diagram of an embodiment of a robotic hand system.

FIG. 4 is a top plan, pictorial view of an embodiment of a robotic hand including a plurality of sensors.

FIG. 5 is a side, elevational pictorial view of an embodiment of a robotic hand in a neutral position.

FIG. 6 is a side, elevational pictorial view of an embodiment of a robotic hand in a grip position.

FIG. 7A is a side, elevational schematic view of an embodiment of an actuator expanding.

FIG. 7B is a side, elevational schematic view of an embodiment of an actuator extending.

FIG. 7C is a side, elevational schematic view of an embodiment of an actuator twisting.

FIG. 7D is a side, elevational schematic view of an embodiment of an actuator bending.

FIG. 8 is a perspective, schematic depiction of an embodiment of an actuator mold.

FIG. 9 is a graphical depiction of force versus time for sustained grip strength of a subject's left hand.

FIG. 10 is a graphical depiction of force versus time for sustained grip strength of a subject's right hand.

FIG. 11 is a graphical depiction of force versus time for a left finger pinch of a subject.

FIG. 12 is a graphical depiction of force versus time for a left full grip of a subject.

### DETAILED DESCRIPTION

As used herein, the term “coupled” can mean two items, directly or indirectly, joined, fastened, associated, connected, or formed integrally together either by chemical or mechanical means, by processes including stamping, molding, or welding, or communicated with a fluid. What is more, two items can be coupled by the use of a third component such as a mechanical fastener, e.g., a screw, a nail, a staple, or a rivet; an adhesive; or a solder.

As used herein, the term “and/or” can mean one, some, or all elements depicted in a list. As an example, “A and/or B” can mean A, B, or a combination of A and B.

Stroke survivors often face challenges in restoring their biomechanical capabilities. Stroke and other events can cause patients to experience difficulties in fine motor skills and muscle strength in their limbs such as their hands, arms, and/or legs. To address this issue, a device can comprise an active soft robotic glove and a rigid passive shoulder support. The soft robotic glove is designed to support motor skills of the hand, while the shoulder support provides stability for the arm when raised. The device is user-friendly, allowing choices of the amount of support the user requires based on the desired application and with a comparatively reduced size and weight, making it suitable for those with biomechanical challenges (e.g., stroke survivors, etc.) with reduced muscle density. The device uses materials like water, compression fabric, and three-dimensional printing filament, making it easily manufacturable and therefore accessible to a wider population of stroke survivors. The results can demonstrate the potential of the soft robotic glove and rigid shoulder support as an effective tool for the rehabilitation and restoration of biomechanical capabilities in the upper extremities of stroke survivors.

The device can be used for any suitable patient having a loss of biomechanical movement or strength due to an injury, such as from a stroke. The injury can create a decreased range of motion in the hand digits and muscle density can be decreased in the hand(s) and arm(s) compared to non-stroke survivors. This difficulty of motion can have an additional impact on the subject because their disuse of the affected side over time can result in a pronounced decrease in muscle density. Decreased muscle density in the shoulder creates difficulty for a subject to hold their arm aloft for a prolonged period of time without experiencing muscle tremors, and decreased muscle density in the hand and forearm makes it difficult for the subject to produce grip strength necessary to hold objects. The device can be used to help perform a variety of tasks including holding a water bottle with one hand hand for opening with the other hand, gripping the handle for pushing a lawnmower, typing on a keyboard, and changing the radio station in the car with one hand while driving with the other. The device described herein can result in at least fifteen percent increase in total grip strength in a subject's hand, fifty percent increase in the subject's ability to hold their arm aloft holding a weight of two pounds, range of motion increase of the subject's hand finger joints by ten percent, and total mechanism weight being under two pounds. This can significantly increase a subject's movement.

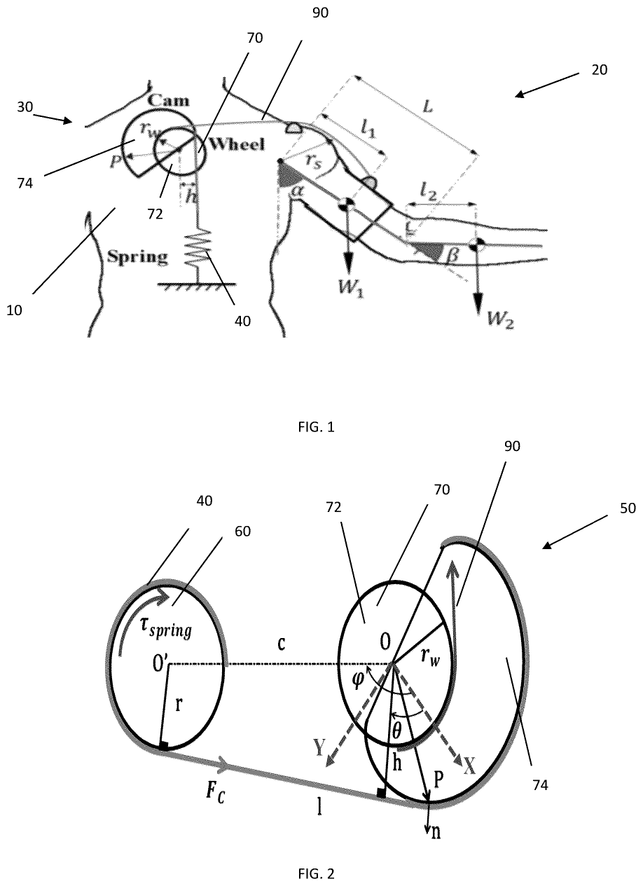

In some embodiments, referring to FIGS. 1-3, a device 20 is provided to a user 10. The device 20 may include an exoskeleton 30, including a preloaded clastic spring 40, a plurality of drums 50, such as a first or storage drum 60 and a second or output drum 70, a cable 90, and/or a robotic hand 100. In some embodiments, the storage drum 60 can store potential energy in the form of the preloaded elastic spring 40. In some aspects, the output drum 70 can include a cam-wheel, specifically a wheel 72 coupled to a cam 74. The cable 90 can be coupled to the user 10, the plurality of drums 50, and the preloaded elastic spring 40.

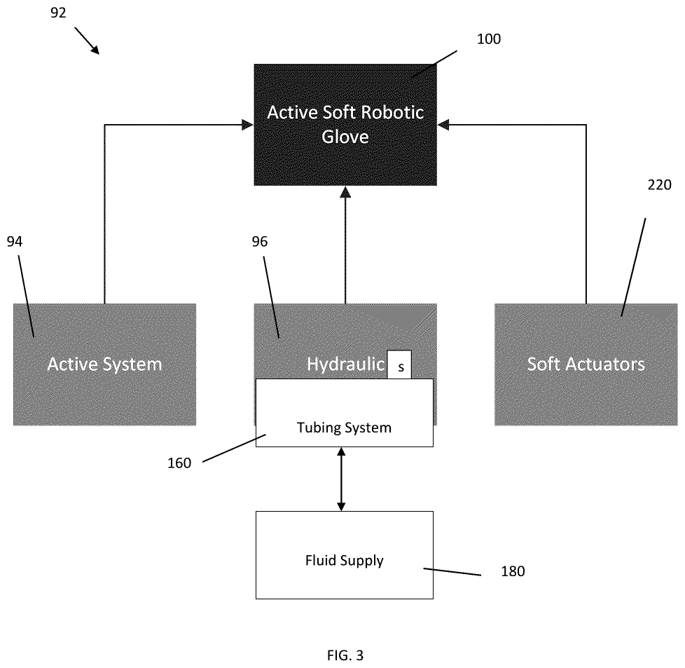

Referring to FIG. 3, in some embodiments a robotic hand system 92 can be included in the device 20. The robotic hand system 92 includes an active system 94, hydraulics 96, and soft actuators, such as a plurality of soft actuators 220, as described in further detail hereinafter. The hydraulics 96 can include a fluid communication system such as a tubing system 160 in fluid communication with a fluid supply 180, which may include any suitable fluid such as an aqueous fluid. In some embodiments, the active system 94 can be utilized as depicted in FIG. 3. An active exoskeleton 30 can use drive systems such as motors, hydraulic systems, or pneumatic systems to enhance human strength or reduce the body's energy consumption. Flex sensors can be used to activate a pump and/or activate a robotic glove 120, as described below.

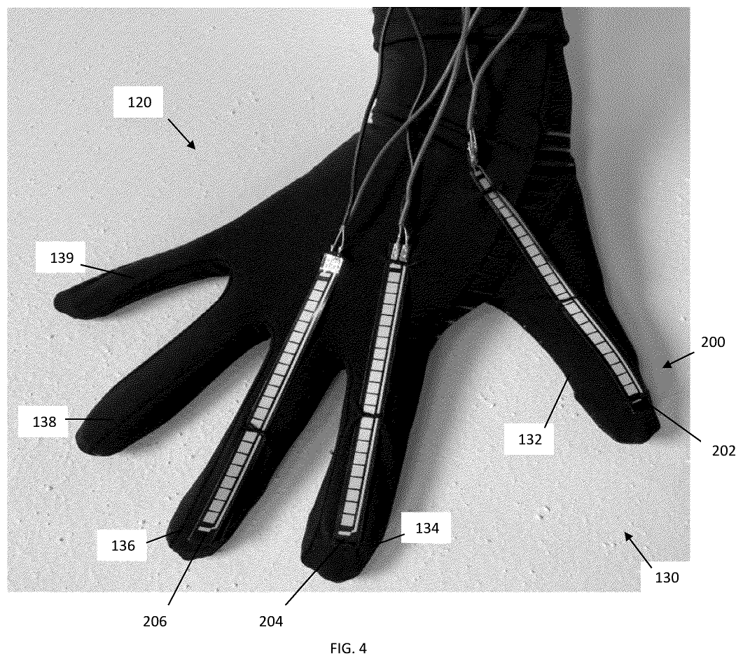

In some embodiments, referring to FIGS. 4-6, the robotic hand 100 can include the glove 120, such as a robotic glove 120, the vinyl tubing system 160 and the fluid supply 180, as depicted in FIG. 3, and a plurality of sensors 200. The glove 120 can include digits 130, such as five digits, including a first digit 132, a second digit 134, a third digit 136, a fourth digit 138, and a fifth digit 139. While five digits are discussed, less than five digits can be included, for example, to provide movement in a select set of fingers or in the fingers as groups. The plurality of sensors 200, can be any suitable number such as three or five sensors. In this embodiment, a first sensor 202, a second sensor 204, and a third sensor 206 are depicted. The first sensor 202 can correspond to the first digit 132, the second sensor 204 can correspond to the second digit 134, and the third sensor 206 can correspond to the third digit 136. In some embodiments, additional sensors can correspond to the fourth digit 138 and the fifth digit 139. The vinyl tubing system 160 can be in fluid communication with the fluid supply 180, as discussed above.

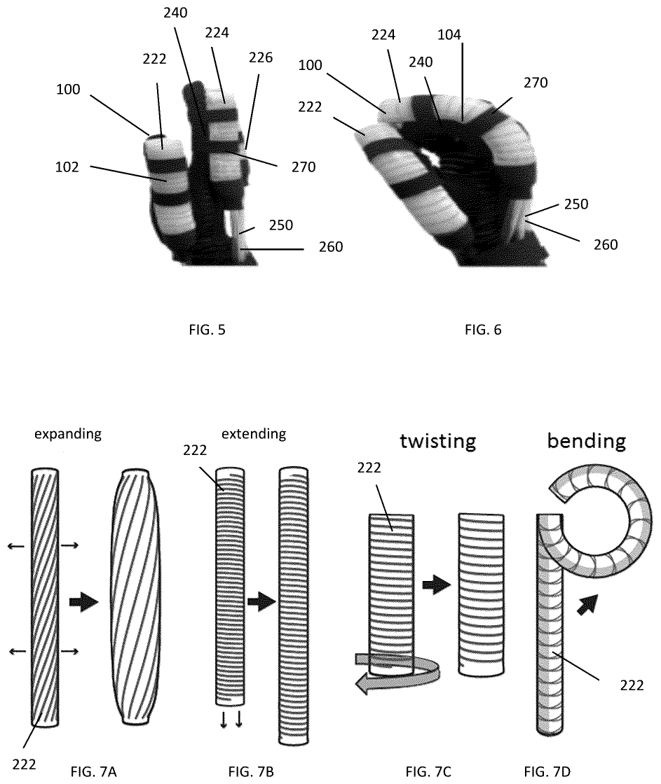

Referring to FIGS. 3-6, a plurality of soft actuators 220, such as a first actuator 222, a second actuator 224, and a third actuator 226 is coupled, correspondingly, to the plurality of 200 sensors. Particularly, the first actuator 222 is coupled to the first sensor 202, the second actuator 224 is coupled to the second sensor 204, and the third actuator 226 is coupled to the third sensor 206. The actuators of the plurality of actuators 220 can be substantially the same, so only the second actuator 224 is described in detail. As depicted in FIGS. 5 and 6, the second actuator 224 may include a thickened base 240, a fluid cavity 250 above the thickened base 240 defined by a tube 260. The retaining device 270 may be wrapped around the tube 260. The robotic hand 100 can be in a neutral position 102 as depicted in FIG. 5 or a grip position 104 as depicted in FIG. 6.

In some embodiments, the device actively assists the user's hand in three supported positions using a hydraulic system. A hydraulic fluid such as water can be used, though some amount of additives can be used in the water. Other hydraulic fluids can also be used. The hydraulic fluid as an incompressible liquid can allow for a higher force output relative to the small actuator size than air or compressible fluids. The flow of the hydraulic fluid can be controlled by a pump such as a peristaltic pump that responds to the force values from flex sensors on the fingers, assisting user motion.

The soft actuators can be made of a top layer of silicone with embedded fiber limits and a bottom strain-limiting layer. In some aspects, the strain-limiting layer can be made of a soft polymer such as silicone with an optional additive such as zinc oxide (ZnO) with a hollow chamber in the middle. As the system is pressurized with water, the strain-limiting segments resist linear actuation of the base, producing a bending motion. In some embodiments, the size of actuators are minimized while still meeting requirements. Efficient geometries of the actuators allow for complex twisting motions and with suitable, stable attachment to the fingers. In some embodiments, the device has efficient geometry for more complex motions, an attachment and grounding, and smaller housing.

Active exoskeletons can be used to drive systems such as motors, hydraulic systems, or pneumatic systems to enhance human strength or reduce the body's energy consumption. Flex sensors and a pump can actuate the robotic glove, and in some aspects, the pump and other components used to actuate the robotic glove can be positioned and supported by the exoskeleton.

One or more soft actuators can be used on the digit portions of a glove. The soft actuators can be hyperelastic and be stimulated by mechanical, thermal, magnetic and electrical energy that can actuate the glove, particularly the digit portions with the first, such as a soft, actuator 222, by expanding, extending, twisting, or bending, as depicted in FIGS. 7A-D.

In some embodiments, the device can result in user having an increase in grip strength of at least about 15%, about 20%, about 25%, about 30%, about 35%, about 40%, about 45%, or about 50%. In some embodiments, the device can have a grip strength of about 15% to about 60%, about 20% to about 55%, about 25% to about 52%, about 30% to about 50%, or about 40% to about 55%.

In some embodiments, the device can result in a user having an increase range of motion of at least about 10%, about 20%, about 30%, about 40%, about 50%, about 60%, about 70%, or about 80%. In some embodiments, the increase range of motion can be about 10% to about 90%, about 30% to about 90%, about 40% to about 90%, or about 50 to about 90%. As an example, a thumb digit of a user can be increased by about 30%, about 40%, about 50%, about 60%, about 70%, or about 80%, a middle digit of a user can be increased by about 20%, about 30%, about 40%, or about 50%, or an index digit of a user can have no increase, or have an increase about 5% or about 10%.

Medical Programs having to deal with rehabilitation therapy for stroke patients working on regaining function can focus on the hand increasing range of motion and grip strength. In some aspects, the glove can be used in addition to traditional physical therapies, with the goal of the glove aiding and possibly expediting the overall process. Furthermore, the glove could even be marketed to individuals as further at home post therapy support with doctors' approval. The marketing and selling of the glove can primarily be through the hospitals and rehab centers handling the therapies of the stroke patients. The glove can be sold directly to the medical institutions and the pharmacies for at home use. The glove can be custom fitted as well as the sizes of the individual components. The glove can improve mobility in the upper body and hand market view.

In some embodiments, this device includes or consists of an active soft robotic glove which assists motor function and dexterity in the hand during grasping, pinching, and pointing motions. The device can use a hydraulic system with soft actuators, and is controlled with biosignals for an intuitive and compliant user interface. This device is suitable for use by people with reduced muscle density due to its compact structure and low weight.

In some embodiments, the device is built on an athletic running glove made of approximately: 41.8% viscose, 29.8% cotton, 23.4% acrylic, and 5% spandex. A vinyl tubing system hooked up to a water supply, a peristaltic pump, tube splitters, and soft actuators are on the glove—the whole system can be controlled by three flex sensors on the thumb, index, and middle finger.

The soft actors can be made of any suitable rubber, such as rubber sold under the trade designation ECO-FLEX® by Smooth-On, Inc. of Macungie, Pennsylvania, and the base of them are thickened with additional zinc oxide. The actuators are also additionally wrapped in embroidery string to act as strain-limiting layers and allow for further actuation.

Some embodiments are unique by being a hydraulic system that utilizes flex sensors over a different control method such as electromyography (EMG). Some embodiments can increase range of motion and grip strength in patients with limited or reduced hand use. The use of a hydraulic system and soft actuators controlled by flex sensors on the hand can control when the glove assists the patient. The increased bulkiness of the hand, the potential for hydraulic system leakage, soft actuator tearing, overall system bulkiness, potential actuation delay, and the difficulty of putting on the glove can affect subject usage. In some embodiments, the geometry can correspond for more complex motions, as well as better attachment and/or grounding to glove base.

In some embodiments, the device can consist or include two distinct parts, the passive mechanical shoulder support or exoskeleton that assists with long-term elevation of the left or right arm and the assistive robotic glove that provides additional grip strength and range of motion in the digits.

The passive shoulder support is designed to assist in prolonged arm extension. During qualitative testing, the patient was asked to extend their arm 90 degrees straight out. They expressed mild tremors in their right arm around the ninety four second mark, and just after four minutes, their arm dropped from exhaustion. The apparatus is a wearable cable-driven passive shoulder exoskeleton that reduces muscle activation in the arm and applies the gravity compensation at the shoulder. This helps with upper extremity tasks to be performed with less effort by the user.

Passive exoskeletons appeal to a wide variety of users because they are lightweight, lower cost, and easy to maintain. Because the passive exoskeleton does not require electromechanical hardware or components, in order to generate forces to assist motions using counterweights, rubber bands or springs. This device encases a preloaded elastic spring that is held by two drums, one for the output energy and the other for storage. The spring force is transmitted by a paracord cable that is strung across the shoulder. This process generates a positive shoulder elevation moment and counteracts the negative movements due to gravity. As the user elevated their arm, the spring is shortened in a clockwise rotation, thus decreasing the spring tension force.

As depicted in FIGS. 1 and 2, showing, respectively, a free body diagram (FBD) of the shoulder support entire device and a free body diagram (FBD) of the cam wheel and spring torque. The elastic spring stretches when the arm is parallel to the user's body and pulls on the paracord to elevate the arm. The elastic elevation moment, coupled with the gravitational moment on the joints when the shoulder is elevated from 0 to 90 degrees.

$${T}_{G} ( α , β ) = {W}_{1} {I}_{1} sin ( α ) + {W}_{2} ( L sin ( α ) + {I}_{2} sin ( ( α , β ) ) \qquad ( 1 )$$

Where W1 is the users arm weight, W2 is the forearm and hand weight added together, l₁ is the distance from the center of the shoulder joint to the center of the user arms gravity, l₂ is the distance from the center of the user's elbow to the hand segment, L is the arm length, and α and β are the shoulder and elbow flexion angles.

On many passive devices the force-generating elements are constant-force springs or rubber bands. Because the shoulder joint has one of the largest ranges of motion, the ideal passive element for the device needs to be able to generate force over a large range of deflection. A constant-force spring was used as the elastic element in this passive device because the spring is able to produce constant force and translate the force over a long distance. In the constant-force spring, a coiled, elastic metal strip is wrapped clockwise around the output drum.

The cam-wheel alters the paracord's pulling force to output the desired positive shoulder elevation with the increasing shoulder angle.

$$φ = \frac{rs}{rw} α \qquad ( 2 )$$

Where φ is the rotation of the cam-wheel, α is the relation of shoulder elevation angle, r_{w} is the constant-radius wheel's radius, and rₛ is the moment of the cable around the shoulder joint.

Generating positive assistive elevation moment throughout the whole system

$${T}_{E} = {kT}_{G} ( α , 0 ) \qquad ( 3 )$$

Where T_{E} is the positive shoulder moment to counteract proportion of (k), k is a constant value of ¼, and T_{G} (α,0) is the gravity moment.

Stability of the cam-wheel balances with assumption of no loss due to friction:

$${F}_{C} ( φ ) h ( φ ) - \frac{rs}{rw} {T}_{E} = 0 \qquad ( 4 )$$

Where FC(φ) is the tension force in the paracord, that wraps around the connections of the constant-force spring.

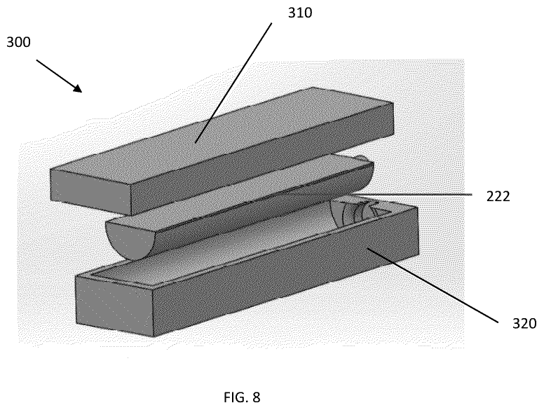

Referring to FIG. 8, an actuator mold 300 is depicted. The actuator mold 300 can include a first, top part 310 and a second, bottom part 320. The actuator mold 300 can be used to fabricate the first actuator 222.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

In this example, the subject receiving the soft robotic hand was invited for multiple rounds of biomedical testing. Data was gathered to customize the solution to the patient, to ensure proper fit and comfort, and to quantitatively validate the device deliverables during its usage.

The first patient benchmark was completed by including physical measurements of the subject, a range of motion evaluation, measurement of instantaneous and average grip strength, basic reaction time and muscle fatigue tests, and a preliminary measurement of surface electromyography responses in the right forearm. First, physical measurements were taken using a standard measuring tape and recorded in centimeters for ease of conversion. The purpose of these measurements was to ensure that the dimensions of the three-dimensional model of the shoulder support, the length of the soft actuators on the digits as well as the connecting tubing, and the size of the compression glove were all sized correctly to the subject. This data is summarized in the table below. All of the measurements relevant to the hand and digits were performed using the right hand only. Below is Table 1 depicting physical measurements of a subject:

TABLE 1

| | |
|---|---|
| Width of | 10.50 |
| shoulders | centimeter |
| | (cm) |
| Radius of | 4.50 cm |
| shoulder | |
| Circumference | 6.40 cm |
| of right arm, just | |
| above wrist bone | |
| Circumference | 6.50 cm |
| of right arm, | |
| just under elbow | |
| Length of thumb | 6.00 cm |
| (base knuckle to | |
| fingertip) | |
| Length of | 8.50 cm |
| index finger | |
| Length of | 8.80 cm |
| middle finger | |
| Length of | 8.35 cm |
| ring finger | |
| Length of | 6.00 cm |
| pinky finger | |
| Length from | 16.50 cm |
| wrist bone to | |
| middle fingertip | |
| Width of hand | 8.80 cm |
| at knuckles | |
| Width at largest | 11.20 cm |
| part of hand | |
| Length of | 27.50 cm |
| forearm, wrist | |
| to elbow | |

Next, a range of motion evaluation was performed using a smartphone application which allows the angle of joints to be superimposed on a picture. Pictures were taken of the index finger at a resting state, full flexion (curling inward, toward the palm), full extension (pointing outward), full adduction (rotating away from the body), and full abduction (rotating inward towards the midpoint of the body). The same was done for the middle, ring, and index digits, as well as the thumb. Next, wrist flexion and wrist extension were measured, as well as elbow flexion and extension. Some interesting and important observations are made. First, the subject's right forearm is not at all able to pronate or supinate (turn upside down, and vice versa). Likewise, the right wrist cannot adduct or abduct. Next, the movement of the index finger and middle finger are linked together and tend to move in unison, as well as the ring and pinkie finger. Most importantly, a significant “mirroring” effect in the fine motion of the digits in the left and right hand is noticed. The left hand appears to “control” some of the motion of the right, and the motion of both hands in unison appear to add additional strength and dexterity to the right hand as opposed to its motion by itself, when the left hand is restricted. The third set of tests performed in this benchmark was the instantaneous and sustained grip strength test, which was performed for both hands. Using a handgrip dynamometer, the subject was instructed to produce maximum grip force for (1) one quick pulse and (2) ten full seconds to evaluate their current capabilities, the difference between the grip strengths in both hands, and as a comparison for later when the subject's grip strength is later improved using the soft robotic glove. The data gathered from the instantaneous phase of grip strength testing is summarized below. Tables 2 and 3 depict, respectively, instantaneous maximum grip strength, left hand and right hand:

TABLE 2

| Left | Trial 1 | Trial 2 | Trial 3 | Trial 4 | Trial 5 | Average |
|---|---|---|---|---|---|---|
| Grip | 250.3 N | 408.2 N | 396.9 N | 354.1 N | 354.7 N | 352.84 N |
| Strength in | | | | | | |
| Newton (N) | | | | | | |

TABLE 3

| Right | Trial 1 | Trial 2 | Trial 3 | Trial 4 | Trial 5 | Average |
|---|---|---|---|---|---|---|
| Grip | 172.3 N | 156.8 N | 116.5 N | 108.2 N | 152.3 N | 141.22 N |
| Strength | | | | | | |
| (N) | | | | | | |

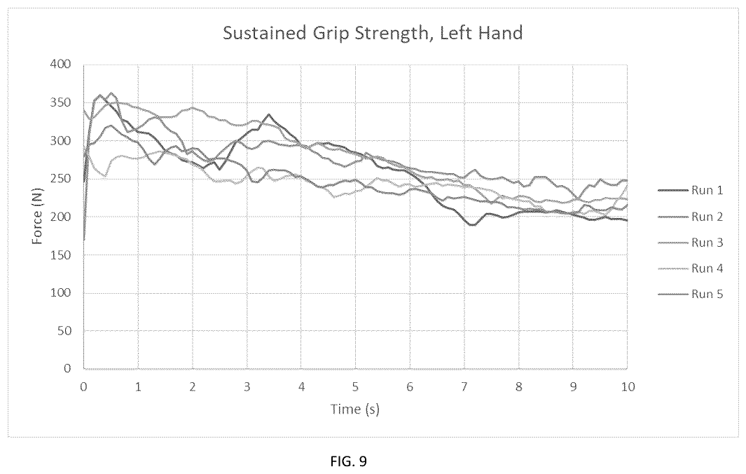

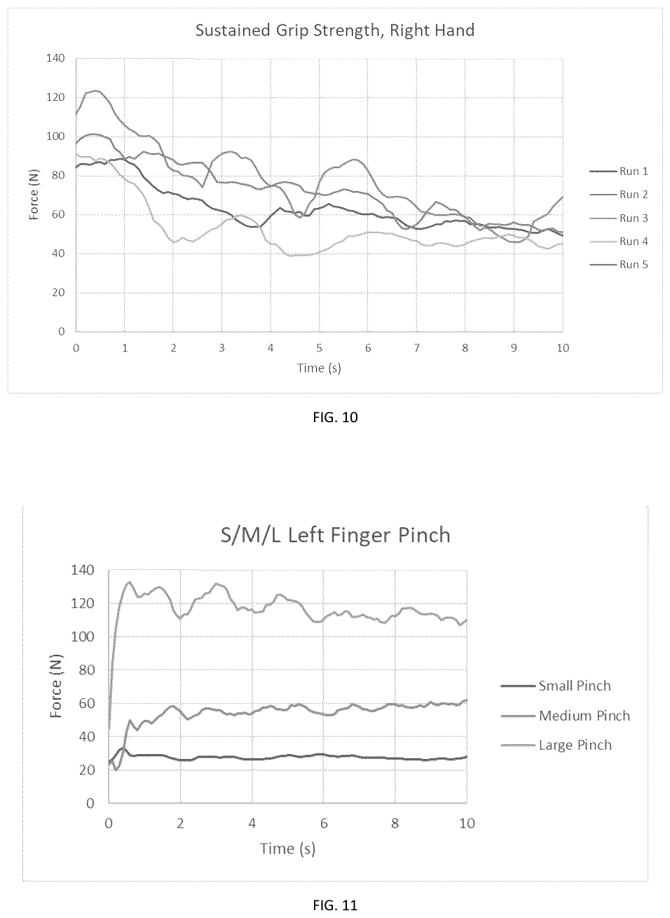

FIGS. 9-10 depict five runs over ten seconds (10 s) depicting sustained grip strength of the left and right hands of a subject. The average of each set of data points was taken and is summarized as an sustained grip strength over 10 s for left and right hands in the Tables 4 and 5 below.

TABLE 4

| | | | | | | Average, |
|---|---|---|---|---|---|---|
| Left Hand | Trial 1 | Trial 2 | Trial 3 | Trial 4 | Trial 5 | all trials |
| Average 10 s | 260.15 N | 275.96 N | 280.79 N | 243.75 N | 246.68 N | 261.46 N |
| Sustained Grip | | | | | | |
| Strength (N) | | | | | | |

TABLE 5

| | | | | | | Average, |
|---|---|---|---|---|---|---|
| Right Hand | Trial 1 | Trial 2 | Trial 3 | Trial 4 | Trial 5 | all trials |
| Average 10 s | 63.03 N | 77.44 N | 66.59 N | 53.14 N | 72.41 N | 66.52 N |
| Sustained | | | | | | |
| Grip | | | | | | |
| Strength (N) | | | | | | |

In the reaction time test, a meter stick was placed so that the zero-meter mark was at the top of the subject's extended, partly closed hand. The stick was dropped without warning and the distance that the meterstick dropped was recorded in centimeters, and then converted using the kinematic equation to equal the amount of time that the subject's hand took to close.

$$\frac{1}{2} {at}^{2} + vt + {x}_{0} = x$$

The kinematic equation used to calculate reaction time, solved for t, is

$$t = \frac{- {v}_{0} \pm \sqrt{{v}_{0}^{2} + 2 ad}}{a}$$

The purpose of this measurement is to determine if there is a significant reduction in muscle activation time in the right hand, and if so, how this would be translated to the soft actuators to allow the glove to grip at the same time as the subject's digits when performing motion. The data for reaction time is summarized in Tables 6 and 7 below:

TABLE 6

| Left | Trial 1 | | Trial 2 | | Trial 3 | | Trial 4 | | Trial 5 | | Trial 6 | | Average | |
|---|---|---|---|---|---|---|---|---|---|---|---|---|---|---|
| Distance of | 15.375 | cm | 18.250 | cm | 9.750 | cm | 9.000 | cm | 12.250 | cm | 11.250 | cm | 12.646 | cm |
| meter stick | | | | | | | | | | | | | | |
| drop (cm) | | | | | | | | | | | | | | |
| Calculated | 0.11714 | s | 0.19299 | s | 0.14106 | s | 0.13553 | s | 0.15811 | s | 0.15152 | s | 0.16065 | s |
| Reaction | | | | | | | | | | | | | | |
| Time | | | | | | | | | | | | | | |
| (s) | | | | | | | | | | | | | | |

TABLE 7

| Right | Trial 1 | | Trial 2 | | Trial 3 | | Trial 4 | | Trial 5 | | Trial 6 | | Average | |
|---|---|---|---|---|---|---|---|---|---|---|---|---|---|---|
| Distance of | 9.750 | cm | 13.750 | cm | 7.750 | cm | 9.875 | cm | 10.825 | cm | 6.125 | cm | 9.679 | cm |
| meter stick | | | | | | | | | | | | | | |
| drop (cm) | | | | | | | | | | | | | | |
| Calculated | 0.14106 | s | 0.16751 | s | 0.12576 | s | 0.14196 | s | 0.14863 | s | 0.11180 | s | 0.14055 | s |
| Reaction | | | | | | | | | | | | | | |
| Time | | | | | | | | | | | | | | |
| (s) | | | | | | | | | | | | | | |

Next, the muscle fatigue test was conducted. The subject was asked to hold their right arm straight out at a 90 degree angle, and instructed to notify the timer when the subject experienced three different phases of fatigue: (1) noticeable discomfort, (2) muscle tremors in the right hand, (3) muscle tremors in the right bicep, and (4) muscle failure, or dropping of the right arm. The purpose of this test is to compare the subject's ability to hold their arm up and the presence of muscle tremors when doing so without support, versus their ability to hold their arm up when supported by the mechanical shoulder device. The subject was only asked to perform this once, as the test was designed to completely exhaust the muscle. The time taken for the subject to report mild discomfort was 94 seconds, the time until muscle tremors in the right hand was 150 seconds, the time to muscle tremors in the right bicep was 230 seconds, and the time to failure was 268 seconds. Lastly, the surface electromyography testing was conducted.

The second patient benchmark was conducted. The three objectives of this meeting were to inform the subject of the design changes in response to the first patient benchmark, have the subject try on the fabric shoulder component and compression glove to evaluate the fit of both, and to do additional grip strength testing on the left hand with the hand dynamometer to establish thresholds for the different amounts of grip strength that the glove should provide. The purpose of this was to evaluate the “normal” grip strength of the subject's unaffected hand in order to be able to mimic a similar amount of grip strength when the hand is supported by the glove using discrete values, e.g. “small”, “medium”, and “large” grip force. These additional left-hand tests were performed similarly to the first grip strength tests; both instantaneously and sustained over a period of 10 seconds. The subject performed these tests with their left hand, first using a full hand grip and secondly using a “pinch” with just their index finger and thumb on opposite sides of the dynamometer.

FIG. 11 depicts pinch data for digits for the left hand of the subject. The average calculated left-handed finger pinch force for a “small” pinch was 27.70 N, force for a “medium” pinch was 54.20 N, and force for a “large” pinch was 116.41 N.

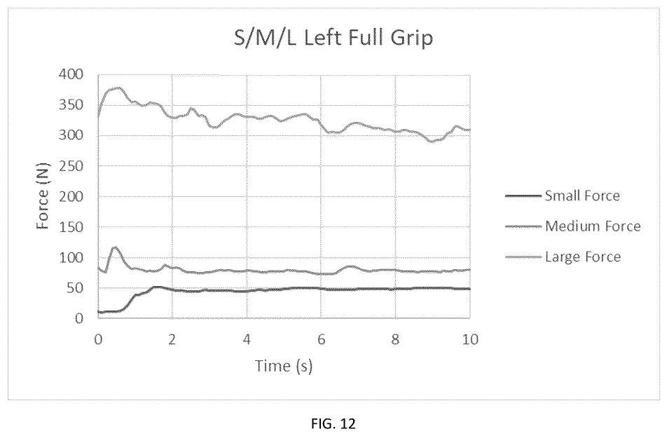

FIG. 12 depicts full grip for the left hand of the subject. The average calculated force of a left-handed “small” grip force was 43.57 N, the average “medium” grip force was 79.23 N, and the average “large” grip force was 326.115 N.

To end the second patient testing session, the subject was asked to try on the fabric component of the shoulder mechanism to test its fit to determine excess fabric.

The third patient benchmark was conducted. In its current state, a Food and Drug Administration (FDA) premarket submission is not required for this device according to the FDA. The device is made of two parts, which when considered separately, can be classified as “Hand, External Limb Component, Powered” (product code IQZ) (FDA 2023) for the glove, and “Assembly, Shoulder/Elbow/Forearm/Wrist/Hand, Mechanical” (product code KFT) (FDA 2023) for the shoulder support mechanism. Both of the aforementioned devices fall under the physical medicine regulation medical specialty, and have been reviewed by a physical medicine review panel which determined that each device is class 1 and is premarket submission exempt. The powered glove is also GMP (“Good Manufacturing Practices”) exempt, but the mechanical shoulder support is not necessarily; it must still uphold the GMP requirements “concerning records (820.180) and complaint files (820.198), as long as the device is not labeled or otherwise represented as sterile”, which it is not. Therefore, for the entire combined device, a premarket submission or an FDA premarket submission is not necessary, but it still must be registered annually with the FDA in order for the product to be marketed commercially in the United States. The manufacturers of this device should still follow FDA guidance and recognized standards for good manufacturing, although it would not be a legal requirement.

Some additional tests were conducted. As an example, a grip strength validation was conducted. The subject's average maximum unassisted grip force was 56.00 N, which was increased using the glove to an average of 84.83, an increase of 51.48%. A one-sided t-test with three trials returned t-value equals 5.233 and p-value equals 0.003184, making the results statistically significant.

In addition, the range of motion (ROM) can be evaluated. The following Table 8 depicts results:

TABLE 8

| | Index digit ROM | Middle digit ROM | Thumb digit ROM |
|---|---|---|---|
| | in degrees | in degrees | in degrees |
| Grasp | 8 to 10.33 | 6 to 9 | 3.67 to 8.33 |
| | Plus 29.17% | Plus 50.00% | Plus 127.27% |
| Pitch | 2.33 to 5, | Not Applicable | 2.67 to 2.67, |
| | Plus 114.29% | | 0.00% |
| Point | Not Applicable | 4 to 2, | 5.33 to 3.33, |
| | | Minus 50.00% | Minus 37.5% |

In this example, depicted in the table above, the total weight of the device is about 0.5 pounds when fully actuated.

As part of rehabilitation therapy for hospitals and rehab centers, at least one glove can be used. Often, the at least one glove is used as part of therapy in conjunction with a doctor's assistance and may also be used as a stand-alone device once the patient has gotten comfortable enough to use the at least one glove independently.

A great deal of research has been conducted on the methods and results of rehabilitation therapy for all kinds of patients experiencing sarcopenia and paralysis in response to a stroke or traumatic injury. As an example, a research study was conducted at the Kumamoto Rehabilitation Hospital in Japan that sought to quantify the relationship between skeletal muscle index (SMI) and functional outcomes in patients experiencing sarcopenia after a stroke, in conjunction with typical rehabilitation methods like range of motion exercises, limb facilitation, resistance training, and activities of daily living (ADL) training, as well as nutritional management.

In some embodiments, the soft robotic glove fulfills all the methods except nutritional management. The elastic properties of the compressive glove help the digits return back to a neutral resting position, moving against the subject's typical, slightly clenched resting position, therefore assisting passively in increasing range of motion over time as the tendons and muscles in the hand learn to relax. The soft actuators perform limb facilitation by moving the subject's digits and applying additional pressure to the grip conformation, helping to train the muscles to perform those actions more effectively over time. The soft actuators also are able to apply different amounts of assistance, which can be changed by the subject using the threshold dial or by changing the code that governs the fill rate of the soft actuators. As the subject learns to utilize the hand effectively and for more daily activities, regaining some degree of muscle function and dexterity over the course of weeks or months, they can decrease this level of assistance. When the level of assistance is decreased, the glove essentially provides resistance training as the subject now has to perform at a greater intensity than before to produce the same actions. Lastly and most importantly, the glove assists with activities of daily life (ADL). The correlated increase in ability to perform ADL with an increase in skeletal muscle mass, because the glove can assist the subject with all activities of daily living, using the glove can also result in an increase of strength for the patient, further acting as a rehabilitative tool.

Having described various systems and methods herein, certain embodiments can include, but are not limited to:

In an aspect, an exoskeleton comprises a preloaded elastic spring; a plurality of drums coupled to the preloaded elastic spring; and a cable coupled to a user, the plurality of drums and the preloaded elastic spring.

A second aspect can include the exoskeleton of the first aspect, wherein the plurality of drums comprises a first drum and a second drum.

A third aspect can include the exoskeleton of the first aspect or the second aspect, wherein the first drum comprises a storage drum and the second drum comprises an output drum.

A fourth aspect can include the exoskeleton of any one of the proceeding aspects, wherein the storage drum has the preloaded elastic spring wrapped thereon and the output drum comprises a cam-wheel.

In a fifth aspect, a robotic hand comprises a glove comprising digits; a vinyl tubing system in fluid communication with a fluid supply; a plurality of sensors coupled to the digits of the glove; and a plurality of soft actuators coupled to the plurality of sensors, wherein the plurality of soft actuators comprises a thickened base, a fluid cavity above the thickened base defined by a tube, and a retaining device wrapped around the tube.

In a sixth aspect, a device comprises an exoskeleton comprising a preloaded elastic spring; a plurality of drums coupled to the preloaded elastic spring; and a cable coupled to a user, the plurality of drums and the preloaded elastic spring; and a robotic hand.

A seventh aspect can include the device of the sixth aspect, wherein the robotic hand can include a glove comprising digits; a vinyl tubing system in fluid communication with a fluid supply; a plurality of sensors coupled to the digits of the glove; and a plurality of soft actuators coupled to the plurality of sensors, wherein the plurality of soft actuators comprises a thickened base, a fluid cavity above the thickened base defined by a tube, and a retaining device wrapped around the tube.

An eighth aspect can include the device of the sixth aspect or seventh aspect, wherein the plurality of drums comprises a first drum and a second drum.

A ninth aspect can include the device of any one of the sixth to eighth aspects, wherein the first drum comprises a storage drum and the second drum comprises an output drum.

A tenth aspect can include the device of any one of the sixth to ninth aspects, wherein the first drum has the preloaded elastic spring wrapped thereon and the second drum comprises a cam-wheel.

An eleventh aspect can include the device of any one of the sixth to tenth aspects, wherein the fluid supply comprises water.

For purposes of the disclosure herein, the term “comprising” includes “consisting” or “consisting essentially of.” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

We claim:

1. An exoskeleton comprising:
a preloaded elastic spring;
a plurality of drums coupled to the preloaded elastic spring; and
a cable coupled to a limb of a user, the plurality of drums and the preloaded elastic spring.

2. The exoskeleton of claim 1, wherein the plurality of drums comprises a first drum and a second drum.

3. The exoskeleton of claim 2, wherein the first drum comprises a storage drum and the second drum comprises an output drum.

4. The exoskeleton of claim 3, wherein the storage drum has the preloaded elastic spring wrapped thereon.

5. The exoskeleton of claim 4, where the output drum comprises a cam-wheel.

6. A device comprising:
a glove comprising digits;
a tubing system in fluid communication with a fluid supply;
a plurality of sensors coupled to the digits of the glove; and
a plurality of soft actuators coupled to the tubing system and the plurality of sensors, wherein the plurality of soft actuators comprises a thickened base, a fluid cavity above the thickened base defined by a tube, and a retaining device wrapped around the tube.

7. The device of claim 6, further comprising:
an exoskeleton comprising a preloaded elastic spring; a plurality of drums coupled to the preloaded elastic spring; and a cable coupled to a user, the plurality of drums and the preloaded elastic spring.

8. The device of claim 7, wherein the plurality of drums comprises a first drum and a second drum.

9. The device of claim 8, wherein the first drum comprises a storage drum and the second drum comprises an output drum.

10. The device of claim 8, wherein the first drum has the preloaded elastic spring wrapped thereon and the second drum comprises a cam-wheel.

11. The device of claim 7, wherein the fluid supply comprises water.

12. A method of movement comprising:
receiving a force input on a sensor of a plurality of sensors on a glove, wherein the glove comprises:
a plurality of digits;
a tubing system in fluid communication with a fluid supply;
the plurality of sensors coupled to the digits of the glove; and
a plurality of soft actuators coupled to the tubing system and the plurality of sensors;
actuating a pump in response to the force input, wherein the pump is in fluid communication with the fluid supply;
transferring fluid between the fluid supply and at least one soft actuator of the plurality of soft actuators; and
applying a force to at least one digit of the plurality of digits in response to the transfer of the fluid.

13. The method of claim 12, further comprising:
applying a force to a limb of a subject using an exoskeleton, wherein the limb is coupled to the at least one digit, and wherein the exoskeleton comprises a preloaded elastic spring; a plurality of drums coupled to the preloaded elastic spring; and a cable coupled to a user, the plurality of drums and the preloaded elastic spring.

14. The method of claim 13, wherein the plurality of drums comprises a first drum and a second drum.

15. The method of claim 14, wherein the first drum comprises a storage drum and the second drum comprises an output drum.

16. The method of claim 14, wherein the first drum has the preloaded elastic spring wrapped thereon and the second drum comprises a cam-wheel.

17. The method of claim 12, wherein the fluid supply comprises water.

18. The method of claim 13, wherein the pump is coupled to the exoskeleton.

19. The method of claim 12, wherein the plurality of soft actuators comprises a thickened base, a fluid cavity above the thickened base defined by a tube, and a retaining device wrapped around the tube.

20. The method of claim 19, wherein applying the force to at least one digit of the plurality of digits in response to the transfer of the fluid comprises transferring fluid into the fluid cavity and applying the force with at least one soft actuator of the plurality of soft actuators.
